(19) United States
(12) Patent Application Publication
(10) Pub. No.: US 2007/0225507 A1
(43) Pub. Date: Sep. 27, 2007

(54) Process for preparing a crystalline form of Tegaserod maleate

(76) Inventors: Gustavo Frenkel, Beer Sheva (IL); Santiago Ini, Haifa (IL)

Correspondence Address: KENYON & KENYON LLP, ONE BROADWAY, NEW YORK, NY, 10004, US

(21) Appl. No.: 11/655,511

(22) Filed: Jan. 18, 2007

Related U.S. Application Data
(60) Provisional application No. 60/760,306, filed on Jan. 18, 2006.
(60) Provisional application No. 60/872,950, filed on Dec. 4, 2006.

(51) Int. Cl. C07D 209/18

(52) U.S. Cl. 548/495

(57) ABSTRACT

Provided is a process for preparing tegaserod maleate.

### RELATED APPLICATIONS

This application claims the benefit of priority to U.S. Provisional Application Nos. 60/760,306, filed Jan. 18, 2006; and 60/872,950, filed Dec. 4, 2006, the contents of which are incorporated herein by reference.

### FIELD OF THE INVENTION

The present invention relates to solid state chemistry of a maleate salt of tegaserod.

### BACKGROUND OF THE INVENTION

Tegaserod maleate is an aminoguanidine indole 5HT4 agonist for the treatment of irritable bowel syndrome (IBS). Tegaserod maleate has the following structure:

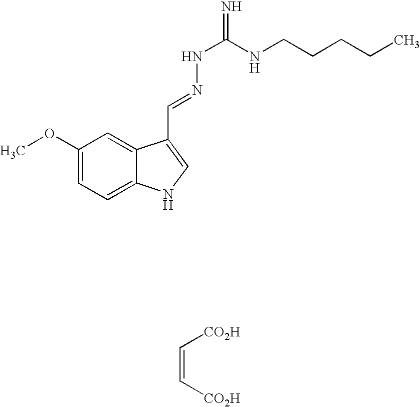

According to the prescribing information (Physician's Desk Reference, 57^{th} Ed., at Page 2339), tegaserod as the maleate salt is a white to off-white crystalline powder and is slightly soluble in ethanol and very slightly soluble in water. Tegaserod maleate is available commercially as ZELNORM®, in which it is present as crystalline form.

Tegaserod maleate is disclosed in U.S. Pat. No. 5,510,353 and in its equivalent EP 0 505 322 (example 13), and is reported to have a melting point of 190° C. (table 1 example 13).

The literature (Buchheit K. H, et al., J. Med. Chem., 1995, 38, 2331) describes a general method for the condensation of aminoguanidines with indole-3-carbadehydes in methanol in the presence of HCl (pH 3-4). The product obtained after solvent evaporation may be converted to its hydrochloride salt by treatment of the methanolic solution with diethylether/HCl followed by recrystallization from methanol/diethylether. Tegaserod base prepared according to this general method is characterized solely by a melting point of 155° C. (table 3 compound 5b). Additional Tegaserod maleate characterization was done by ¹H and ¹³C-NMR according to the literature (Jing J. et. al., Guangdong Weiliang Yuansu Kexue, 2002, 9/2, 51).

The solid state physical properties of tegaserod salt may be influenced by controlling the conditions under which tegaserod salt is obtained in solid Form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state Form of a compound may also affect its behavior on compaction and its storage stability.

These practical physical characteristics are influenced by the conformation and orientation of molecules in the unit cell, which defines a particular polymorphic Form of a substance. The polymorphic form may give rise to thermal behavior different from that of the amorphous material or another polymorphic Form. Thermal behavior is measured in the laboratory by such techniques as capillary melting point, thermogravimetric analysis (TGA) and differential scanning calorimetry (DSC) and may be used to distinguish some polymorphic forms from others. A particular polymorphic Form may also give rise to distinct spectroscopic properties that may be detectable by powder X-ray crystallography, solid state C NMR spectrometry and infrared spectrometry.

WO 04/085393 discloses four crystalline forms of tegaserod maleate. The search report for WO 04/085393 further identifies WO 00/10526, and Drugs Fut. 1999, 24(1) which provides an overview for tegaserod maleate. Additional crystalline forms of tegaserod maleate are provided in WO 2005/058819.

One of the crystal forms disclosed in WO 2005/058819 is designated as Form B, which is characterized by an X-ray Diffraction pattern having peaks at 15.7, 16.9, 17.2, 24.1, 24.6 and 25.2±0.2 two theta. In that publication, Form B is prepared by slurrying solid tegaserod maleate in an alcohol. There is a need in the art for a one pot process suitable for industrial scale that prepares Form B from tegaserod base without isolation of tegaserod maleate as an intermediate.

### SUMMARY OF THE INVENTION

In one embodiment the present invention provides a one-pot process comprises reacting N-amino-N′-pentylguanidine hydroiodide (“AGP-HI”) and 5-Methoxy-1H-indole-3-carbaldehyde (“MICHO”) in an aqueous reaction mixture to obtain tegaserod base; extracting the tegaserod base with a water immiscible organic solvent to obtain a mixture; combining the mixture with n-propanol to obtain a slurry; and combining the slurry with maleic acid to obtain tegaserod maleate Form B. The water immiscible solvent is preferably ethyl acetate.

### BRIEF DESCRIPTION OF THE FIGURES

FIG. 1 illustrates an X-ray powder diffraction pattern for tegaserod maleate Form B according to example 1.

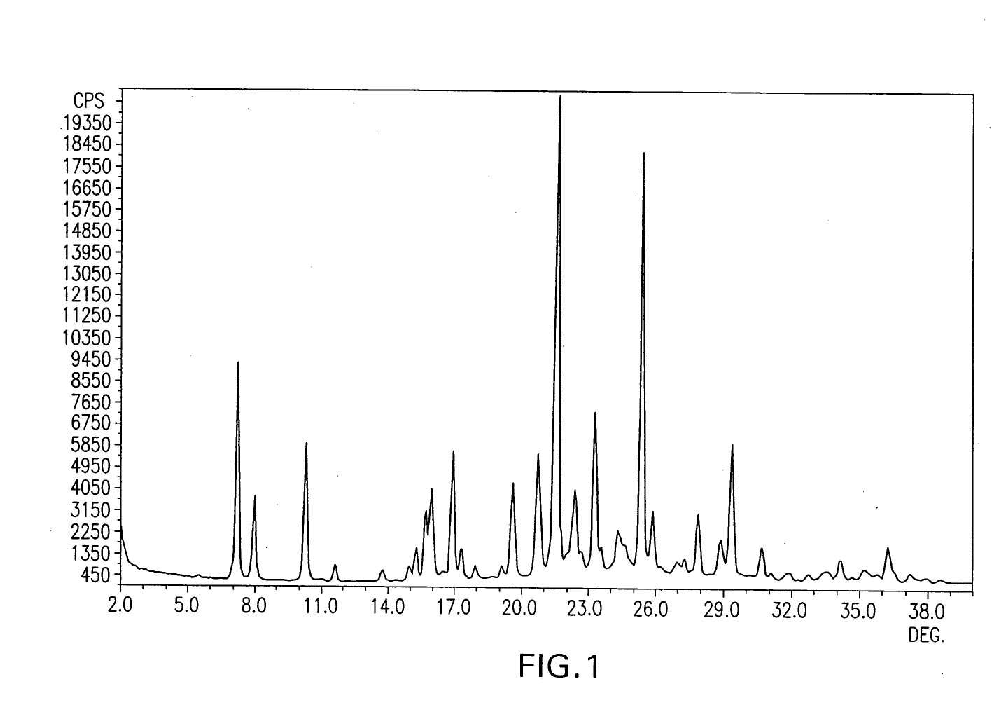

### DETAILED DESCRIPTION OF THE INVENTION

The present invention provides a one pot process for preparing the tegaserod maleate crystalline form characterized by an X-ray Diffraction pattern having peaks at 15.7, 16.9, 17.2, 24.1, 24.6 and 25.2±0.2 two theta (designated as Form-B). The one pot process is ideal for use on industrial scale because it avoids isolation of another crystalline form of maleate as an intermediate.

This one-pot process comprises reacting N-amino-N′-pentylguanidine hydroiodide (“AGP-HI”) and 5-Methoxy-1H-indole-3-carbaldehyde (“MICHO”) in an aqueous reaction mixture to obtain tegaserod base; extracting the tegaserod base with a water immiscible organic solvent to obtain a mixture; combining the mixture with n-propanol to obtain a slurry; and combining the slurry with maleic acid to obtain tegaserod maleate Form B.

The reaction of AGP-HI with MICHO may be carried out under acidic or basic conditions. When the reaction is carried out under aqueous basic conditions an organic or inorganic base may be used. The organic base is preferably a C₃ to C₈ alkyl amine such as trialkylamine (preferably triethylamine), and pyridine. The inorganic base may be an alkali/alkaline earth-hydroxide or carbonate, preferably K₂CO₃, Na₂CO₃, NaHCO₃, NaOH, KOH, more preferably NaOH. The reaction is preferably carried out at a pH range of 7 to 14, more preferably of about 9 to 14. The temperature range during the reaction is preferably of about 5° C. to reflux temperature. When a tertiary amine is used, the tertiary amine may also act as a solvent, thus, the reaction may be carried out in the presence of the tertiary amine in neat form, i.e. without the use of an additional solvent.

When the reaction is carried out under acidic conditions water is added, and an organic or inorganic acid may be used. An organic acid such as p-toluensulfonic acid, pyridinium p-toluenesulfonic acid, methanesulfonic acid, acetic acid or maleic acid may be used. In another embodiment, an inorganic acid such as HCl, HBr, H₃PO₄ or H₂SO₄ may be used. The pH range during the reaction is preferably of 1 to 7, more preferably of about 3 to 4. The temperature range during the reaction is preferably of about 5° C. to about reflux temperature of water Before conversion to the maleate salt, a base may be used to neutralize the acid used in the process or to eliminate undesirable salts.

In one embodiment, an aqueous solution of AGP-HI is combined with a mixture of MICHO and a solid base. Alkali and alkaline earth metal bases, such as potassium and sodium hydroxide may be used.

The tegaserod formed during the reaction can be extracted into a water immiscible organic solvent. Preferably, the water immiscible organic solvent is selected from the group consisting of: C₃-C₇ esters, C₃₋₈ ethers, C₃₋₇ ketones and dimethyl sulfoxide (DMSO). More preferably, the water immiscible organic solvent is a C₃-C₇ ester, most preferably ethyl acetate. In one embodiment, ethyl acetate is mixed with the tegaserod containing reaction mixture, after which tegaserod moves to the ethyl acetate. The reaction mixture may be stirred to accelerate the extraction process. The organic phase may then be washed with an aqueous solvent such as water to remove water miscible impurities. The organic phase may also be filtered to further remove impurities.

N-propanol is then added to the reaction mixture. N-propanol can be added either alone or as a mixture with another solvent, preferably ethyl acetate. The final ratio of ethyl acetate to n-propanol is preferably about 1:1 to about 1:3 (v/v). The reaction mixture may be heated, preferably of about room temperature to about 70° C., such as about 60° C. to about 65° C. Maleic acid, either neat or as a solution in water, is combined with the reaction mixture. The resulting mixture may then be stirred, such as for about I to about 12 hours. The resulting reaction mixture can be cooled to precipitate Form B. Cooling can be carried out to a temperature of about 5° C. to about room temperature.

Tegaserod maleate Form B may be recovered from the reaction mixture by any method known in the art, such as filtering. The tegaserod maleate may further be washed. Preferably, Form B is washed with n-propanol. The tegaserod maleate may also be dried. The temperature may be increased and/or pressure reduced to accelerate the drying process. The drying process may be carried out at a temperature of about 35° C. to about 55° C., preferably about 45° C. The pressure may be that of a vacuum, i.e., a pressure of less than about 100 mmHg. A vacuum oven can be used.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

PXRD:

Powder X-ray diffraction (“PXRD”) analysis using a SCINTAG powder X-ray diffractometer model X′TRA equipped with a solid-state detector. Copper radiation of λ=1.5418 Å was used. The sample was introduced using a round standard aluminum sample holder with round zero background quartz plate in the bottom.

Thermal Gravimetric Analysis (TGA):

TGA/SDTA 851^{e}, Mettler Toledo, Sample weight 7-15 mg. Heating rate: 10° C./min., in N₂ stream: flow rate: 50 ml/min

### Example 1

Preparation of Tegaserod Maleate Form B

To a mixture of 90 g MICHO and 63 g NaOH [47%] was added a solution of 212 g AGP·HI dissolved in 566 mL of water at room temperature. The resultant reaction mixture was heated to 40° C. After 3 hours, 522 mL of ethyl acetate was added and the reaction mixture was stirred for an additional hour. The organic phase was washed with water (3×450 mL), and vacuum filtered. After addition of 211 mL ethyl acetate and 870 mL of n-propanol, the mixture was heated to 60° C. and a solution of maleic acid (86.5 g in 180 mL water), at the same temperature, was added to the reaction mixture and stirred at the same temperature. After 2 hours the reaction mixture was cooled to about 10° C. and stirred for an additional hour. The resulting solid was filtered off, washed with n-propanol, and dried in a vacuum oven over night to give 195.8 g of tegaserod maleate Form B.

What is claimed is:

1. A one-pot process for preparation of tegaserod maleate Form B comprising reacting N-amino-N′-pentylguanidine hydroiodide (“AGP-HI”) and 5-Methoxy-1H-indole-3-carbaldehyde (“MICHO”) in an aqueous reaction mixture to obtain tegaserod base; extracting the tegaserod base with a water immiscible organic solvent to obtain a mixture; combining the mixture with n-propanol to obtain a slurry; and combining the slurry with maleic acid to obtain tegaserod maleate Form B.

2. The process of claim 1, wherein the reaction of AGP-HI with MICHO is carried out under acidic or basic conditions.

3. The process of claim 1, wherein pH during the reaction is from above 7 to about 14.

4. The process of claim 3, wherein pH during the reaction is about 9 to about 14.

5. The process of claim 3, wherein an aqueous solution of AGP-HI is combined with a mixture of MICHO and a solid base.

6. The process of any one of claims 3, wherein an alkali or alkaline earth metal base is used.

7. The process of claim 6, wherein the base is selected from the group consisting of K₂CO₃, Na₂CO₃, NaHCO₃, NaOH and KOH,

8. The process of claim 3, wherein an organic base is used.

9. The process of claim 8, wherein the organic base is a C₃ to C₈ alkyl amine or pyridine.

10. The process of claim 8, wherein the base is triethylamine

11. The process of claim 10, wherein the organic base acts as a neat reagent.

12. The process of claim 1, wherein the pH is about 3 to about 7.

13. The process of claim 12, wherein the pH is about 3 to about 4.

14. The process of claim 1, wherein an organic acid is used during the reaction.

15. The process of claim 14, wherein the organic acid is selected from the group consisting of p-toluensulfonic acid, pyridinium p-toluenesulfonic acid, methanesulfonic acid, acetic acid, maleic acid and mixtures thereof.

16. The process of claim 1, wherein an inorganic acid is used during the reaction.

17. The process of claim 16, wherein the inorganic acid is selected from the group consisting of HCl, HBr, H₃PO₄, H₂SO₄ and mixtures thereof.

18. The process of claim 1, wherein the reaction mixture is heated to accelerate the reaction.

19. The process of claim 18, wherein the temperature range during the reaction is about 5° C. to reflux temperature.

20. The process of claim 19, wherein heating is carried out to a temperature of about 35° C. to about 45° C.

21. The process of claim 1, wherein the reaction between AGP-HI and MICHO is maintained for about 30 minutes to about 24 hours.

22. The process of claim 21, wherein the reaction between AGP-HI and MICHO is maintained for about 2 hours to about 4 hours.

23. The process of claim 1, wherein the tegaserod formed during the reaction is extracted into ethyl acetate, followed by addition of n-propanol before combining with maleic acid.

24. The process of claim 23, wherein the n-propanol is added as a mixture with ethyl acetate.

25. The process of claim 1, wherein the water immiscible organic solvent is selected from the group consisting of: C₃-C₇ esters, C₃₋₈ ethers, C₃₋₇ ketones and dimethyl sulfoxide (DMSO).

26. The process of claim 25, wherein the water immiscible organic solvent is a C₃-C₇ ester.

27. The process of claim 26, wherein the solvent is ethyl acetate.

28. The process of claim 1, wherein ratio of ethyl acetate (water-immiscible solvent) to n-propanol is about 1:1 to about 1:3 (v/v).

29. The process of claim 1, wherein a solution of maleic acid in water is combined with the reaction mixture.

30. The process of claim 1, wherein the reaction mixture is cooled to precipitate the crystalline form.

31. The process of claim 30, wherein cooling is carried out to a temperature of about 5° C. to about room temperature.

32. The process of claim 1, further comprising recovering the crystalline form.

33. The process of claim 32, further comprising washing the crystalline form with n-propanol.

34. The process of claim 32, further comprising drying the crystalline form.

35. The process of claim 34, wherein drying is carried out at a temperature of about 35° C. to about 55° C.

36. The process of claim 35, wherein the temperature is about 45° C.

37. The process of claim 35, wherein drying is carried out at a pressure of less than about 100 mmHg.
